(19) United States
(12) Patent Application Publication
(10) Pub. No.: US 2023/0148996 A1
(43) Pub. Date: May 18, 2023

(54) LUNG ULTRASOUND PROCESSING SYSTEMS AND METHODS

(71) Applicant: ALVEOLAI DATAFUEL INC., London (CA)

(72) Inventors: Robert Thomas ARNTFIELD, London (CA); Blake VANBERLO, London (CA); Derek WU, London (CA); Benjamin WU, London (CA); Jared TSCHIRHART, Simcoe (CA); Chintan DAVE, London (CA); Joseph MCCAULEY, London (CA); Alex FORD, London (CA); Scott MILLINGTON, Ottawa (CA); Jordan HO, London (CA); Rushil CHAUDHARY, London (CA); Jason DEGLINT, Waterloo (CA); Thamer ALAIFAN, London (CA); Nathan PHELPS, London (CA); Matthew WHITE, London (CA)

(21) Appl. No.: 18/099,206

(22) Filed: Jan. 19, 2023

Related U.S. Application Data
(63) Continuation of application No. PCT/CA2021/050995, filed on Jul. 19, 2021.

(51) Int. Cl. A61B 8/08; A61B 8/00

(52) U.S. Cl. CPC A61B 8/5223; A61B 8/08; A61B 8/4427; A61B 8/5207

(57) ABSTRACT

Methods and systems for processing data to distinguish between a plurality of conditions in lung ultrasound images and, in particular, lung ultrasound images containing B lines. Neural network systems and methods, in which the processor is trained using lung ultrasound images to distinguish between acute respiratory distress syndrome due to COVID-19, acute respiratory distress syndrome due to non-COVID-19 causes, and hydrostatic pulmonary edema.

### CROSS-REFERENCE TO RELATED APPLICATION

This application is a continuation of International Application No. PCT/CA2021/050995, filed Jul. 19, 2021, which claims the benefit of and priority to U.S. Provisional Application No. 63/054,169, filed Jul. 20, 2020, the entireties of each of which are incorporated herein by reference.

### FIELD

The described embodiments relate to processing of medical imaging data, and in particular to systems and methods for processing of lung ultrasound data.

### INTRODUCTION

The following is not an admission that anything discussed below is part of the prior art or part of the common general knowledge of a person skilled in the art.

### SUMMARY

The following summary is provided to introduce the reader to the more detailed discussion to follow. The summary is not intended to limit or define any claimed or as yet unclaimed invention. One or more inventions may reside in any combination or sub-combination of the elements or process steps disclosed in any part of this document including its claims and figures.

In a broad aspect, there is provided a method of processing data to distinguish between a plurality of conditions based on at least one ultrasound image of a lung, the method comprising: providing the at least one ultrasound image of the lung; preprocessing the at least one ultrasound image to produce a tensor; processing the tensor using a neural network to produce an intermediate tensor; down-sampling the intermediate tensor to produce an output tensor; and processing the output tensor using an output neural network to generate a probability of presence of a first condition of the plurality of conditions in the at least one ultrasound image.

In some embodiments, the preprocessing may further comprise converting the at least one ultrasound image to grayscale.

In some cases, the at least one ultrasound image is a plurality of ultrasound images forming a video, and wherein the preprocessing further comprises selecting a still image from the video for use in the tensor.

In some cases, the neural network is a depthwise separable convolutional neural network. In some cases, the neural network is based on an Xception neural network.

In some cases, the tensor has dimensions 512×512×3.

In some cases, the downsampling comprises performing two-dimensional global average pooling, and wherein the output tensor is one-dimensional.

In some cases, the output neural network is a 3-layer fully connected network with softmax activation.

In some cases, generating the probability further comprises generating an output classification vector, wherein the output classification vector represents a plurality of probabilities of each of the plurality of conditions.

In some cases, the first condition is acute respiratory distress syndrome due to COVID-19. In some cases, a second condition of the plurality of conditions is acute respiratory distress syndrome due to non-COVID-19 causes. In some cases, a third condition of the plurality of conditions is hydrostatic pulmonary edema.

In some cases, the neural network is configured to activate in response to the presence of one or more subvisible artifact in the at least one ultrasound image.

In some cases, the one or more subvisible artifact is located in a pleural line region of the at least one ultrasound image.

In some cases, the at least one ultrasound image contains B-lines.

In some embodiments, the method may further comprise determining that the probability of the first condition exceeds a predetermined threshold.

In some embodiments, the method may further comprise isolating a subject in accordance with a triage protocol in response to determining that the probability of the first condition exceeds the predetermined threshold.

In some embodiments, the method may further comprise applying a first treatment to a subject in response to determining that the probability of the first condition exceeds the predetermined threshold.

In some cases, the at least one ultrasound image is obtained via a point-of-care ultrasound device.

In some embodiments, the method may further comprise prior to processing the tensor, pre-training the neural network to obtain pre-trained weights by performing the obtaining the at least one ultrasound image, the preprocessing, the processing the tensor, the downsampling and the processing the output tensor, wherein, during pre-training, the at least one ultrasound image is obtained from an image database. In some cases, the image database is the ImageNet database.

In some embodiments, the method may further comprise discarding a first portion of the neural network following the pre-training.

In some embodiments, the method may further comprise training the neural network to obtain trained weights by performing the obtaining the at least one ultrasound image, the preprocessing, the processing the tensor, the downsampling and the processing the output tensor, wherein, during training, the at least one ultrasound image is obtained from a validated ultrasound image dataset.

In some cases, the training is performed using an Adam optimizer with a learning rate of 1×10−6. In some cases, the training is performed with early stopping with a patience of 3 epochs conditioned on validation loss. In some cases, during training, the downsampling further comprises applying dropout at a rate of about 0.6. In some cases, during training, the preprocessing further comprises performing an augmentation transformation to the at least one ultrasound image. In some cases, the augmentation transformation is selected from the group consisting of: random zooming by up to about 10%; horizontal flipping; horizontal stretching or contraction by up to about 20%; vertical stretching or contraction by up to about 5%; or rotation by up to about 10°.

In some embodiments, the method may further comprise generating a visualization that depicts activations of the final convolutional layer of the neural network to identify relative contributions of each portion of the at least one ultrasound to the probability.

In some cases, the visualization is a gradient heatmap. In some cases, the gradient heatmap is resampled to an original size of the at least one ultrasound image, and overlaid onto the at least one ultrasound image, to produce a validation image.

In some embodiments, the method may further comprise obtaining the at least one ultrasound image of the lung from a subject.

In some embodiments, the method comprising initially analyzing the at least one ultrasound image using an A-line versus B-line deep learning classifier to determine whether the at least one ultrasound image corresponds to a pathological class.

In some embodiments, the classifier is a network that includes a modified VGG16 architecture with weights pre-trained on ImageNet.

In some embodiments, the method further comprises training the A-line versus B-line deep learning classifier by: receiving a plurality of training lung ultrasound clips; labelling the plurality of training lung ultrasound clips to generate a plurality of labelled clips; pre-processing the plurality of labelled clips to generate pre-processed clips; and training a deep learning model using the plurality of labelled clips.

In some embodiments, the labelling comprises labelling each of the plurality of training clips with the presence of either A-lines or B-lines.

In some embodiments, labelling clips with the presence of B-lines comprises labelling the clips as one of: (a) having fewer than three B-lines in the field, (b) having B-lines occupying less than 50% of the pleural line, and (c) having B-lines occupying more than 50% of the pleural line surface.

In some embodiments, the pre-processing comprises de-constructing each of the plurality of lung ultrasound clips into their respective constituent frames, and in respect of each frame, applying one or more transformations.

In some embodiments, the transformations applied to a frame comprise one or more of: rotation of the frame by up to a 45° clockwise or counter clockwise rotation, a vertical or horizontal width shift by up to 10%, magnification by up to 10% inwardly or outwardly, shearing by up to 10° counter clockwise, horizontal reflection, or increasing or decreasing brightness by up to 30%.

In some embodiments, the method further comprises resizing the frames to 128×128×3 tensors.

In some embodiments, the training comprises feeding the pre-processed frame as input tensors with dimensions of 128×128×3 and passing the input tensors through an initial three blocks of the VGG16 model, wherein the initial three blocks comprise ten layers and each block comprises convolution layers followed by a max pooling layer.

In some embodiments, the method further comprises passing a 32×32×256 output of a final convolution layer of the VGG 16 model to a 2D global average pooling layer.

In some embodiments, training the deep learning model comprises a feature extraction phase and a fine-tuning phase.

In some embodiments, the feature extraction phase comprises freezing the ten VGG16 layers of the initial three blocks and training the final layer for six epochs using an Adam optimizer with a learning rate of 0.0003.

In some embodiments, the fine-tuning phase comprises training weights in (i) the third block, of the initial three blocks, and (ii) the output fully connected layer, for nine epochs using an RMSProp optimizer at a learning rate of 9.3×10⁻⁶, while keeping the remaining two blocks, of the of the initial three blocks, stagnant.

In another broad aspect, there is provided a non-transitory computer readable medium storing computer program instructions which, when executed by at least one processor, cause the at least one processor to carry out the methods substantially as described herein.

In still another broad aspect, there is provided a system for processing data to distinguish between a plurality of conditions based on at least one ultrasound image of a lung, the system comprising: a memory; and at least one processor configured to carry out the methods substantially as described herein. In some cases, the system may further comprise a point-of-care ultrasound device configured to obtain the at least one ultrasound image from the subject.

It will be appreciated by a person skilled in the art that a system, method or computer readable medium disclosed herein may embody any one or more of the features contained herein and that the features may be used in any particular combination or sub-combination.

These and other aspects and features of various embodiments will be described in greater detail below.

### BRIEF DESCRIPTION OF THE DRAWINGS

The drawings, described below, are provided for purposes of illustration, and not of limitation, of the aspects and features of various examples of embodiments described herein. It will be appreciated that for simplicity and clarity of illustration, where considered appropriate, reference numerals may be repeated among the drawings to indicate corresponding or analogous elements or steps.

A preferred embodiment of the present invention will now be described in detail with reference to the drawings, in which:

FIG. 1 is three sample images and lung ultrasound characteristics typical of three lung pathologies distinguished in accordance with at least some of the described embodiments;

FIG. 2 is a process flow diagram illustrating a data acquisition, selection and verification workflow in accordance with some embodiments;

FIG. 3 is a process flow diagram illustrating an example process for model selection and validation in accordance with some embodiments;

FIG. 4 illustrates performance plots over a number of epochs from a selection of training experiments for a plurality of neural network models;

FIG. 5 is a simplified schematic block diagram illustrating a processing architecture in accordance with at least some embodiments;

FIG. 6 illustrates performance plots over three classes of images;

FIG. 7 illustrates test images and the corresponding heatmaps for two example images in each of three classes of image;

FIGS. 8A to 8E are example images illustrating stages of a masking approach for an ultrasound image in accordance with some embodiments;

FIG. 9 is a histogram displaying the results of a Monte Carlo simulation as described further herein;

FIG. 10 is another schematic diagram of the structure of the modified Xception neural network architecture in accordance with some embodiments;

FIG. 11 is a process flow diagram for a method of processing data to distinguish between a plurality of conditions of a lung in accordance with at least some embodiments;

FIG. 12 is a process flow diagram for a method for determining whether a lung ultrasound clip is part of a normal class or an abnormal class;

FIG. 13 is a process flow diagram for a method of training and implementing an A-line versus B-line classifier for lung ultrasound clips; and

FIG. 14 is a plot showing clip-wise labelling performance at various thresholds of contiguous B-lines on example data.

### DESCRIPTION OF VARIOUS EMBODIMENTS

Various systems or methods will be described below to provide an example of an embodiment of the claimed subject matter. No embodiment described below limits any claimed subject matter and any claimed subject matter may cover methods or systems that differ from those described below. The claimed subject matter is not limited to systems or methods having all of the features of any one system or method described below or to features common to multiple or all of the apparatuses or methods described below. It is possible that a system or method described below is not an embodiment that is recited in any claimed subject matter. Any subject matter disclosed in a system or method described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a pushbutton keyboard, mouse, a touchscreen, and the like), and at least one output device (e.g. a display screen, a printer, a wireless radio, and the like) depending on the nature of the device.

It should also be noted that there may be some elements that are used to implement at least part of one of the embodiments described herein that may be implemented via software that is written in a high-level computer programming language such as object oriented programming. Accordingly, the program code may be written in Python, Go, Java, C, C++ or any other suitable programming language and may comprise modules or classes, as is known to those skilled in object oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage.

Lung ultrasound (LUS) is an imaging technique deployed by clinicians at the point-of-care to aid in the diagnosis and management of acute respiratory failure. LUS provides chest X-ray (CXR)-level or better diagnostic performance for most acute respiratory illnesses (see, e.g., Long L, Zhao H-T, Zhang Z-Y, Wang G-Y, Zhao H-L, Lung ultrasound for the diagnosis of pneumonia in adults: A meta-analysis, Medicine, 2017 January; 96(3):e5713; Lichtenstein D A, Mezière G A, Relevance of lung ultrasound in the diagnosis of acute respiratory failure the BLUE protocol, Chest, 2008; 134(1):117-25; and Ma O J, Mateer J R, Trauma ultrasound examination versus chest radiography in the detection of hemothorax, Ann Emerg Med, 1997 March; 29(3):312-5; discussion 315-6), LUS lacks the radiation and laborious workflow of computed tomography (CT) scanning. Further, as a low cost, battery operated tablet or smartphone compatible modality, LUS can be delivered at large scale in any environment and is ideally suited for pandemic conditions (see, e.g., Buonsenso D, Pata D, Chiaretti A, COVID-19 outbreak: less stethoscope, more ultrasound, Lancet Respir Med, 2020 May; 8(5):e27).

B lines are the characteristic, vertical, hyperechoic artifact seen on LUS images that is created by either pulmonary edema or non-cardiac causes of interstitial syndromes. The latter includes a broad list of conditions ranging from pneumonia, pneumonitis, acute respiratory distress syndrome (ARDS), pulmonary contusion or fibrosis (see, e.g., Dietrich C F, Mathis G, Blaivas M, Volpicelli G, Seibel A, Wastl D, et al, Lung B-line artefacts and their use, J Thorac Dis, 2016 June; 8(6):1356-6), each of which may require different treatment approaches. While an accompanying thickened pleural line is helpful in differentiating cardiogenic from non-cardiogenic causes of B lines (see, e.g., Copetti R, Soldati G, Copetti P, Chest sonography: A useful tool to differentiate acute cardiogenic pulmonary edema from acute respiratory distress syndrome, Cardiovasc Ultrasound [Internet], 2008; Available from: http://dx,doi, org/10,1186/1476-7120-6-16), reliable methods to differentiate non-cardiogenic causes from one another in LUS images have not been established previously. Additionally, user dependent interpretation of LUS contributes to a wide variation in disease classification (see, e.g., Corradi F, Via G, Forfori F, Brusasco C, Tavazzi G, Lung ultrasound and B-lines quantification inaccuracy: B sure to have the right solution, Intensive Care Med, 2020 May; 46(5):1081-3; and Millington S J, Arntfield R T, Guo R J, Koenig S, Kory P, Noble V, et al, Expert Agreement in the Interpretation of Lung Ultrasound Studies Performed on Mechanically Ventilated Patients, J Ultrasound Med, 2018 November; 37(11):2659-65). At least some of the described embodiments may provide greater precision and minimize user-dependence, thereby improving the usefulness of LUS.

Deep learning (DL), a class of artificial intelligence (AI) techniques, has been shown to meet or exceed clinician performance across most visual fields of medicine (see, e.g., Chilamkurthy S, Ghosh R, Tanamala S, Biviji M, Campeau N G, Venugopal V K, et al, Deep learning algorithms for detection of critical findings in head CT scans: a retrospective study, Lancet [Internet], 2018; Available from: http://dx,doi, org/10,1016/S0140-6736(18)31645-3; Gulshan V, Peng L, Coram M, Stumpe M C, Wu D, Narayanaswamy A, et al, Development and validation of a deep learning algorithm for detection of diabetic retinopathy in retinal fundus photographs, JAMA—Journal of the American Medical Association [Internet], 2016; Available from: http://dx,doi,org/10,1001/jama,2016,17216; Brinker T J, Hekler A, Enk A H, Kl ode J, Hauschild A, Berking C, et al, Deep learning outperformed 136 of 157 dermatologists in a head-to-head dermoscopic melanoma image classification task, Eur J Cancer [Internet], 2019; Available from: http://dx,doi, org/10,1016/j,ejca,2019,04,001). Without cognitive bias or reliance on spatial relationships between pixels, DL ingests images as numeric sequences and evaluates for quantitative patterns that may reveal information that is unavailable to the human, qualitative analysis (see, e.g., Poplin R, Varadarajan A V, Blumer K, Liu Y, McConnell M V, Corrado G S, et al. Prediction of cardiovascular risk factors from retinal fundus photographs via deep learning. Nature Biomedical Engineering [Internet]. 2018; Available from: http://dx.doi.org/10.1038/s41551-018-0195-0). With CT and CXR research maturing (see, e.g., Dean N, Irvin J A, Samir P S, Jephson A, Conner K, Lungren M P. Real-time electronic interpretation of digital chest images using artificial intelligence in emergency department patients suspected of pneumonia. Eur Respir J [Internet]. 2019 Sep. 28 [cited 2020 Jul. 3]; 54(suppl 63). Available from: https://erj.ersjournals.com/content/54/suppl 63/0A3309; Li L, Qin L, Xu Z, Yin Y, Wang X, Kong B, et al. Artificial Intelligence Distinguishes COVID-19 from Community Acquired Pneumonia on Chest CT. Radiology [Internet]. 2020; Available from: http://dx.doi.org/10.1148/radio1.2020200905; Song Y, Zheng S, Li L, Zhang X, Zhang X, Huang Z, et al. Deep learning Enables Accurate Diagnosis of Novel Coronavirus (COVID-19) with CT images. medRxiv [Internet]. 2020; Available from: http://dx.doi.org/10.1101/2020.02.23.20026930), deep learning has shown favourable results in recent CXR and CT studies of COVID-19 (see Apostolopoulos I D, Mpesiana T A, Covid-19: automatic detection from X-ray images utilizing transfer learning with convolutional neural networks, Australas Phys Eng Sci Med, 2020 June; 43(2):635-40; and Nouvenne A, Zani M D, Milanese G, Parise A, Baciarello M, Bignami E G, et al, Lung Ultrasound in COVID-19 Pneumonia: Correlations with Chest CT on Hospital admission, Respiration, 2020 Jun. 22; 1-8).

LUS image creation is fundamentally different from CXR and CT images, producing artifacts rather than anatomic images of the lung. Therefore, currently it has not been made clear that adequate results can be achieved with DL processing of LUS images. In particular, LUS remains comparably understudied with DL, perhaps due to a paucity of organized, well-labelled LUS data sets and the seeming lack of rich information in its minimalistic, artifact-based images. However, LUS enjoys advantages over CT and CXR approaches, since LUS images (unlike CT or CXR) can be obtained by personnel with a variety of abilities, at low cost and in a variety of locations.

The described embodiments generally differ from other LUS-related artificial intelligence work. Lung ultrasound artifact analysis has been available in some commercial ultrasound systems and has also been described using various methods in the literature (see, e.g., Brusasco C, Santori G, Bruzzo E, Trò R, Robba C, Tavazzi G, et al, Quantitative lung ultrasonography: a putative new algorithm for automatic detection and quantification of B-lines, Crit Care, 2019 Aug. 28; 23(1):288; and Corradi F, Brusasco C, Vezzani A, Santori G, Manca T, Ball L, et al, Computer-Aided Quantitative Ultrasonography for Detection of Pulmonary Edema in Mechanically Ventilated Cardiac Surgery Patients, Chest, 2016 September; 150(3):640-51). Automating the detection of canonical findings of LUS, these techniques are convenient and serve to achieve what clinicians may be trained to do with minimal training (see, e.g., Lim J S, Lee S, Do H H, Oh K H, Can Limited Education of Lung Ultrasound Be Conducted to Medical Students Properly? A Pilot Study, Biomed Res Int, 2017 Mar. 28; 2017:8147075). With attention to COVID-19, Roy et al have applied DL techniques to correlate LUS image features that align with a proposed clinical severity score for COVID (see, e.g., Roy S, Menapace W, Oei S, Luijten B, Fini E, Saltori C, et al, Deep learning for classification and localization of COVID-19 markers in point-of-care lung ultrasound, IEEE Trans Med Imaging [Internet], 2020; Available from: http://dx,doi, org/10,1109/tmi,2020,2994459). In a pre-printed article, Born et al trained a classifier to distinguish between three classes of lung findings that are known to be distinguishable by the human eye: B lines, consolidation and A lines (see Born J, Brändle G, Cossio M, Disdier M, Goulet J, Roulin J, et al, POCOVID-Net: Automatic Detection of COVID-19 From a New Lung Ultrasound Imaging Dataset (POCUS), arXiv [Internet], 2020 Apr. 25; Available from: http://arxiv,org/abs/2004,12084).

The focus on prognostic markers in COVID LUS images is, however, distinct from the concept of determining the presence of subvisible artifacts or features within the pixels of the LUS images (as compared to the NCOVID and HPE groups), as in at least some of the described embodiments.

In particular, at least some of the described embodiments demonstrate that deep learning techniques may be capable of detecting patterns, i.e., subvisible artifacts, at levels that exceed human vision. When coupled with the advantages of lung ultrasound (LUS) technology, the pattern recognition abilities of deep learning may provide the ability to distinguish between similar appearing lung diseases. The described embodiments may generally provide systems and methods for discriminating between conditions that produce characteristic B lines on LUS, including COVID-19, using a neural network classifier model on an annotated LUS data repository.

At least some of the described embodiments can use labelled LUS data to train a neural network (e.g., convolutional neural network) using three similar-appearing LUS B line patterns emanating from three distinct pathological processes: 1) hydrostatic pulmonary edema (HPE), 2) acute respiratory distress syndrome (ARDS) due to non-COVID causes (NCOVID), and 3) ARDS due to COVID-19 (COVID). Non-COVID causes may be, e.g., related to any or all of Influenza A, Influenza B, para-influenza, meta-pneumovirus, streptococcal, staphylococcal organisms, etc. At least one example embodiment was trained and validated, and followed by two test phases, including one test phase using a hold-back set of an additional 12,370 still images (10% of the available image data) to validate results. Example embodiments were further validated by benchmarking human ability for comparison purposes, using a LUS interpretation survey of ultrasound-competent physicians. To facilitate explainability, a Grad-CAM approach was used to generate visualizations.

The trained neural network of at least one example embodiment distinguished the underlying pathology in the three sets of pathological B lines. On the hold-back validation set, the example embodiment was capable of discriminating between COVID (AUC 1.0), NCOVID (AUC 0.934) and pulmonary edema (AUC 1.0) encounters, where AUC means “area under the receiver operating characteristic curve” as described further below. Human physician benchmarking provided comparative results of AUC 0.697 for COVID, AUC 0.704 for NCOVID, and AUC 0.967 for HPE, with p<0.01.

Training data was drawn from a database of point-of-care ultrasound (POCUS) exams of the lung performed at London Health Sciences Centre's two tertiary hospitals. The methods of curation and oversight of this archive are described in Arntfield RT, The utility of remote supervision with feedback as a method to deliver high-volume critical care ultrasound training, J Crit Care [Internet], 2015; Available from: http://dx,doi,org/10,1016/j,jcrc,2014,12,006.

At least some of the described embodiments can use neural networks trained to classify images with obvious qualitative differences (HPE vs ARDS) and with no obvious differences (NCOVID vs COVID) between their B lines patterns.

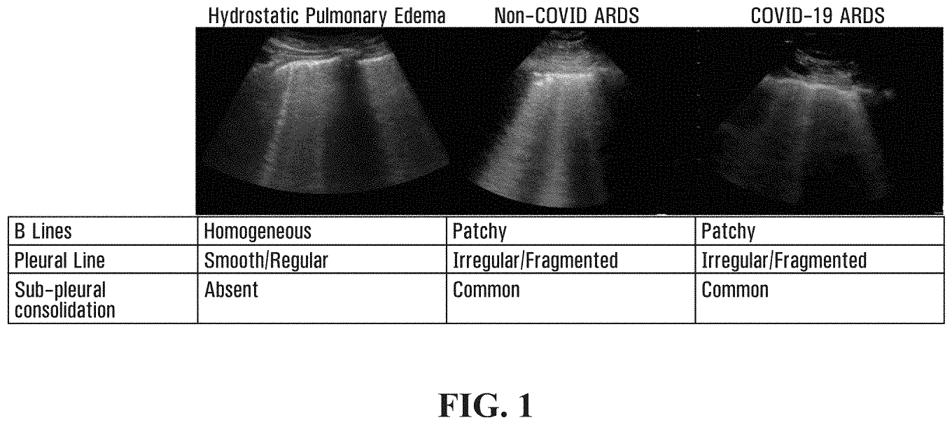

FIG. 1 illustrates three sample images and lung ultrasound characteristics typical of the three lung pathologies at least some of the described embodiments were trained to distinguish. It can be seen that, while the image showing HPE pathology is distinguishable based on the homogeneity of the B lines, the smoothness or regularity of the pleural line and the absence of sub-pleural consolidation, these features do not permit distinguishing between COVID-related ARDS and non-COVID ARDS.

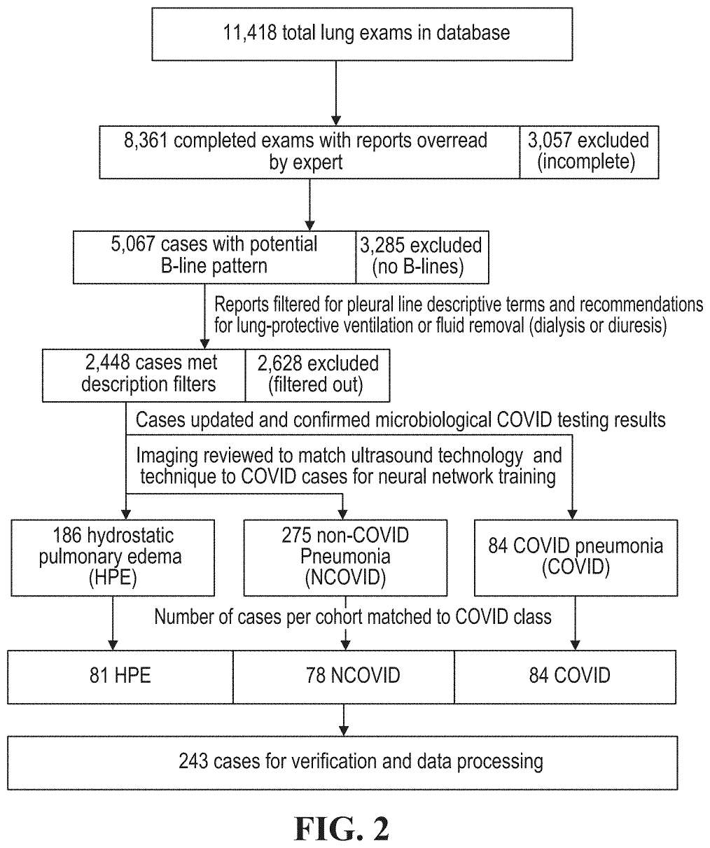

Candidate images for inclusion were identified using a sequential search by two critical care physicians, both trained in ultrasound techniques, from within the finalized clinical reports of the database of LUS cases. FIG. 2 illustrates an example data acquisition, selection and verification workflow.

Videos from the LUS case database represented a variety of ultrasound systems with a phased array probe predominantly used for acquisition. Videos of the costophrenic region (which included solid abdominal organs, diaphragm, or other pleural pathologies such as effusions or trans-lobar consolidations) were excluded as 1) these regions did not contribute greatly to alveolar diagnoses, 2) this would introduce heterogeneity into the still image data, and 3) a trained clinician can easily distinguish between these pathologies and B lines. Duplicate studies were discarded to avoid overfitting. From each encounter, de-identified MPEG-4 video loops of B lines, ranging from 3-6 seconds in length with a frame rate ranging from 30-60/second (depending on the ultrasound system), were extracted. As COVID is the newest class available in the database, there was a comparably smaller number of encounters, as compared to HPE and NCOVID classes. However, using a balanced volume of data for each class of image may assist in avoiding model over training on a single image class and/or overfitting.

The images used to train the model may be frames from the extracted LUS clips. Hereafter, a clip refers to a LUS video that consists of several image frames. An encounter is considered to be a set of one or more clips that were acquired during the same LUS examination.

To build the dataset, each clip may be broken into frames and saved as image files. Frames from clips of the same encounter may be grouped together in a folder for that encounter.

Preprocessing of each frame may consist of a conversion to grayscale followed scrubbing the image of extraneous information (index marks, logos, and manufacturer-specific user interface).

For example, in order to remove extraneous information, the ultrasound beam may be isolated from the rest of the image by a mask. The masking approach may be generalizable to ultrasound beams of varying widths and positions, as different machines have different interfaces. To facilitate finding a suitable mask, the largest continuous contour may be detected using computer vision techniques for a plurality of frames in a video, and the frame with the largest contour may be used for calculations of the ultrasound beam. The two linear edges may be derived by finding every point on the contour that is both the top-most and left- or right-most in every column and row, and finding two lines of best fit for both sets of points. The bottom circular edge may then be calculated using the intersection of these two lines as the vertex, and fitted to all the bottom-most points on the contour. A mask may be generated using these edges and subsequently applied to every frame in the video, as all frames in the same video may have the beam in the same position. If a matching ultrasound beam is not found within empirically determined limits due to poor-quality beams, then the process may revert to using the contour as the mask. This approach may facilitate removing information outside of the ultrasound beam and preserving information that may have been left out by the contour. In some cases, however, text or interface artifacts contained within the beam portion of the image may retained.

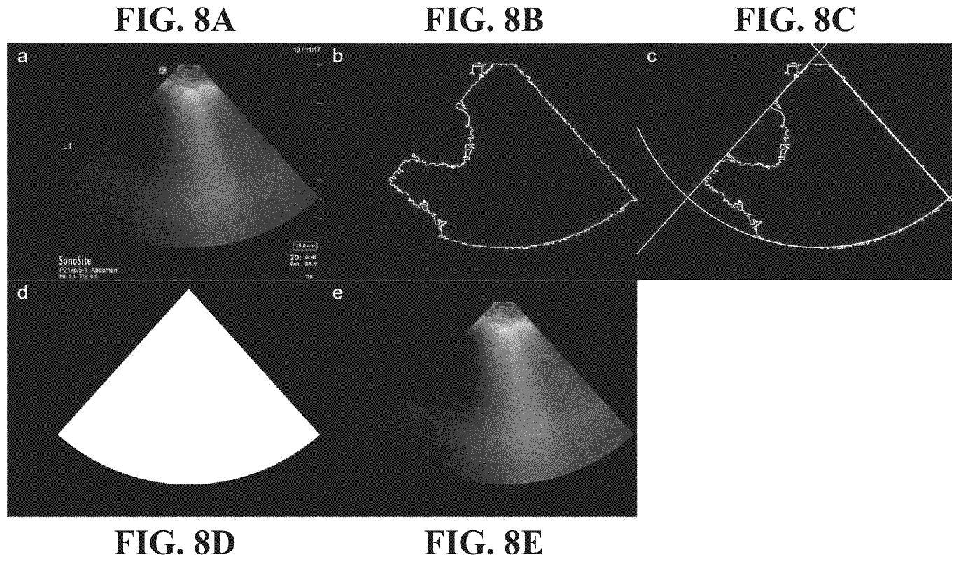

FIGS. 8A to 8D illustrate the masking approach for an ultrasound image. FIG. 8A illustrates the original frame; FIG. 8B illustrates the detected contour of the ultrasound beam; FIG. 8C illustrates the edges as calculated; FIG. 8D illustrates the resulting mask region; and FIG. 8E illustrates the final pre-processed frame.

In some cases, data augmentation techniques may be applied to images in each batch of training data during training to prevent overfitting (see, e.g., Perez L, Wang J, The Effectiveness of Data Augmentation in Image Classification using Deep Learning [Internet], arXiv [cs,CV], 2017, Available from: http://arxiv,org/abs/1712,0462). Augmentation transformations may include random zooming in/out by ≤10%, horizontal flipping, horizontal stretching/contracting by ≤20%, vertical stretching/contracting (≤5%), and bi-directional rotation by ≤10°.

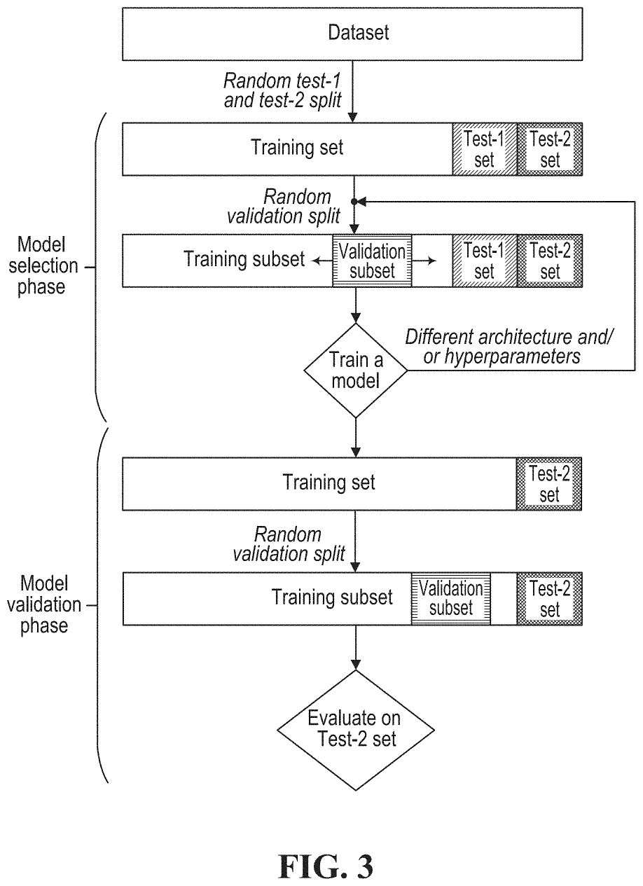

In at least some embodiments, a neural network based on the Xception model may be used. However, in other embodiments, other models may be used. For example, feedforward CNNs and residual CNNs may be used, along with transfer learning (TL) methods. FIG. 3 illustrates an example process for model selection and validation.

Transfer learning is a technique that utilizes previously learned weights from a neural network trained on a separate problem and has previously shown success with CNNs for other ultrasound classification problems (see, e.g., Byra M, Styczynski G, Szmigielski C, Kalinowski P, Michalowski L, Paluszkiewicz R, et al, Transfer learning with deep convolutional neural network for liver steatosis assessment in ultrasound images, Int J Comput Assist Radiol Surg [Internet], 2018; Available from: http://dx,doi,org/10,1007/s11548-018-1843-2).

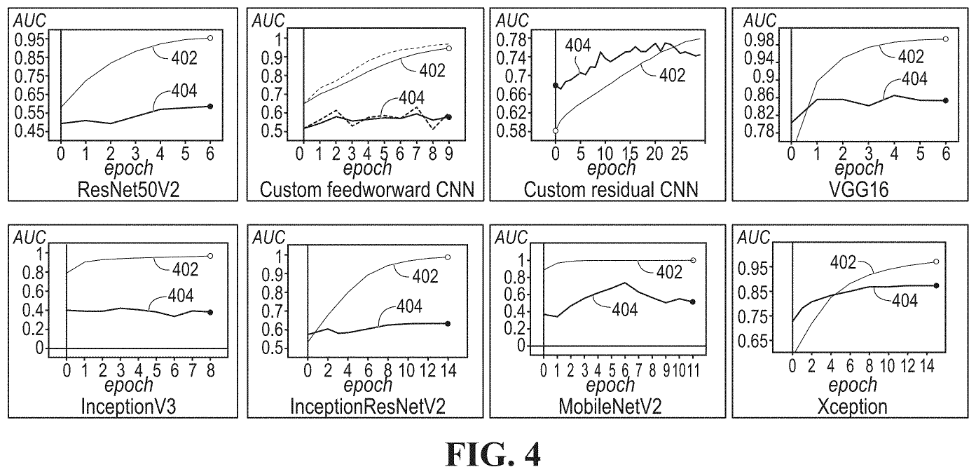

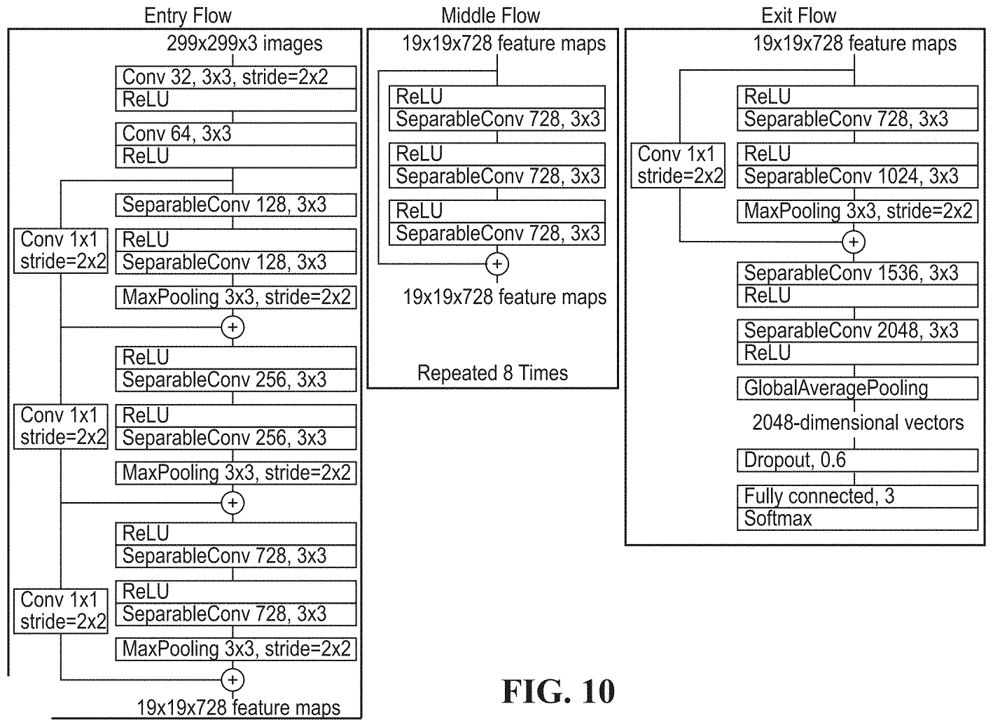

In the described embodiments, a neural network model based on Xception was chosen following initial TL trials that involved fine-tuning several architectures, including included ResNet50V2, ResNet101V2, VGG16, InceptionV3, InceptionResNetV2, MobileNetV2 and Xception. Xception is a CNN architecture that employs depth wise separable convolutions to amplify training efficiency (see, e.g., Chollet F, Xception: Deep learning with depthwise separable convolutions, In: Proceedings—30th IEEE Conference on Computer Vision and Pattern Recognition, CVPR 2017 [Internet], 2017, Available from: http://dx,doi, org/10,1109/CVPR,2017,195). In training experiments, a modified Xception model achieved the highest performance on different validation subsets and on the test-1 set, as shown in FIG. 4. The modified Xception architecture was further fine-tuned with differing configurations of the model head and sets of hyperparameters. FIG. 10 illustrates a schematic diagram of the structure of the modified Xception neural network architecture. In particular, the modified Xception architecture takes the conventional Xception model, excludes the “head” and adds a selection of layers. A detailed specification of one example modified Xception model, with the intermediate dimensions of the input as it flows through the model layers, is provided in Appendix A.

Referring now to FIG. 4, there are shown plots of the AUC for each epoch from a selection of training experiments. In each plot, the line 402 represents training performance while the line 404 represents the validation performance. In each plot, the upper- and right-most extent of the line 402 terminates above the line 404.

In the described embodiments, the initial weights can be pre-trained on the ImageNet dataset available at https://image-net.org, and publicly available through the TensorFlow machine learning platform at https://www.tensorflow.org. ImageNet is a benchmark database of real-world images containing images annotated with approximately 1000 classes. In some cases, weights for selected models—pre-trained for image classification—can be downloaded from, e.g., the TensorFlow project and used. This allows for the model, such as one based on Xception, to be pre-trained to identify features lower abstraction, such as edges and simple shapes. Subsequently, pre-trained weights from the model “head” can be excluded. Individual preprocessed images can be fed into the network as a tensor with dimensions 512×512×3. The output tensor of the final convolutional layer of the modified Xception model can be subject to downsampling, such as, 2D global average pooling, to produce a 1-dimensional tensor. In some cases, dropout at a rate of around 0.6 can be applied to introduce heavy regularization to the model and provide a reduction in overfitting. The final layer can be a 3-node fully connected layer with softmax activation. The output of the model can thereby represent the probabilities that the model assigned to each of the three classes (e.g., HPE, COVID, NCOVID), all summing to 1.0. The argmax of this probability distribution can be considered to be the model's decision.

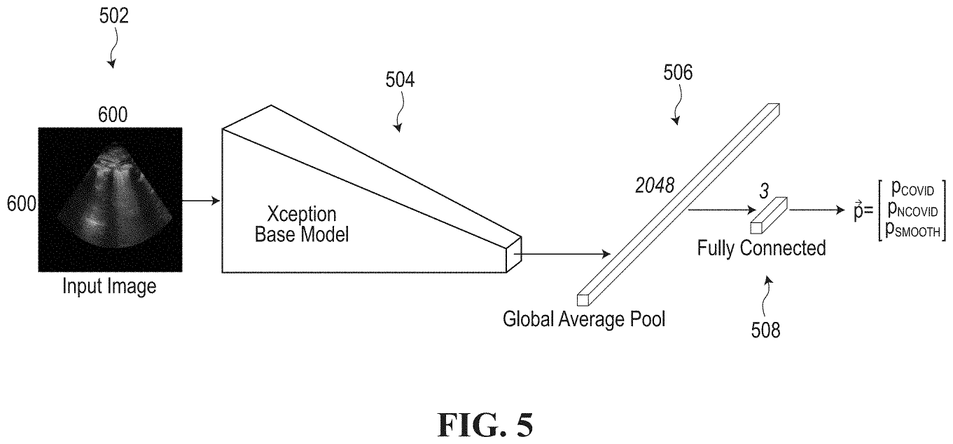

Referring now to FIG. 5, there is illustrated a schematic diagram of the system in accordance with at least some described embodiments. As shown in FIG. 5, the system ingests an input ultrasound image 502 to the convolutional base of the modified Xception model 504. Global average pooling 506 can be performed on the output from the convolutional base of the modified Xception model. Finally, a fully connected layer 508 with softmax activation can output prediction probabilities for each class.

In at least some embodiments, the neural network (e.g., CNN) can be trained using the Adam optimizer with a learning rate (a) of 1×10⁻⁶ to minimize the binary cross-entropy loss function. The magnitude of the loss function for any particular prediction during training can be weighted by the representation in the dataset of each class. In some cases, early stopping with a patience of 3 epochs can be applied, conditioned on validation loss, as a means of regularization (see, e.g., Prechelt L, Early Stopping—But When? In: Montavon G, Orr G B, Müller K-R, editors, Neural Networks: Tricks of the Trade: Second Edition, Berlin, Heidelberg: Springer Berlin Heidelberg; 2012, p, 53-67).

In at least some embodiments, software code may be written in the Python programming language, such as Python version 3.7. However, other programming environments may also be used. In at least some embodiments, the TensorFlow open source machine learning platform (www.tensorflow.org) may be used to define and train the CNN model, however other platforms may also be used.

In at least some embodiments, a computer system may be used for training and processing as described herein. The computer system generally has at least one processing element, such as central processing unit (CPU) and may include one or more specialized processing elements, such as graphics processing units (GPUs), neural processing elements (NPUs) and the like. The computer system generally also has volatile and/or non-volatile memory, and may have input and output devices such as network interfaces, keyboards, touch input devices, pointing devices, graphic displays and the like. In at least one example embodiment, hardware used for training included a personal computer running Microsoft® Windows® 10, equipped with an Inter® Core i9-9900K processor at 3.6 GHz and a NVIDIA® GeForce® RTX 2080 Ti GPU with 11 GB of memory. Three virtual machines (VMs) were employed for training experiments. Each VM ran Ubuntu 18.04 LTS Server. The VMs provided access to NVIDIA® Tesla T4 GPUs, each with 16 GB of memory and Tensor Cores. In the example embodiment, one VM had 5 such GPUs and the others had 3. In at least embodiments, the data types of some variables in the neural network model may be set to 16-bit floating-point to optimize the use of the Tensor Cores on the T4 GPUs.

For validation purposes, a modification of the holdout validation method can be used to facilitate that the iterative model selection process is independent of the model validation. The holdout approach may begin with an initial split that randomly or pseudo randomly partitions all encounters into a training set and two test sets (henceforth referred to as test-1 set and test-2 set as shown in FIG. 3). The distribution of encounters and frames after this split for one example validation are shown in Table 1. Splits are generally conducted at the encounter level, not across all frames, to facilitate that frames from the same clip did not appear in more than one partition. Test-2 may be considered to be the holdout set, since it remains untouched throughout the model selection process and is generally only used during the final validation phase.

TABLE 1
Distribution of encounters and frames assigned to each dataset

| Data Split | Encounters [% of total] | Frames [% of total] |
|---|---|---|
| Training set | 204 | 99471 |
| Test-1 set | 19 (7.82%) | 9540 (7.86%) |
| Test-2 set | 20 (8.23%) | 12370 (10.19%) |

At the start of each training experiment, the training set may be randomly split into a training subset and a validation subset. As a result, the validation subset may differ in each successive experiment. Since early stopping can be employed, the validation subset may be involved in the model selection process. After each training experiment, the model can be assessed based on its performance on the validation subset and the test-1 set. In this way, test-1 can be used for model selection with the dynamic validation subsets.

Once a model architecture has been selected with a particular set of hyperparameters, the model validation phase may begin. To train the final model, the training set and test-1 can be combined to form a larger training set, which can be subsequently split into a training and validation subset for the final experiment. Once training is complete, the trained model can be evaluated by calculating performance metrics from its predictions on the examples in the test-2 set.

In at least some of the described embodiments, a selected model was evaluated based on a holdout test set (test-2) to provide an unbiased estimate of the model's generalization performance. The results were analyzed both at the individual frame level and at the encounter level. The latter was achieved through averaging the classifier's predicted probabilities across all images from within that encounter. The model's performance was assessed by calculating the area under the receiver operating characteristic curve (AUC), analyzing a confusion matrix, and calculating metrics derived from the confusion matrix.

Benchmarking human performance for comparison to a given model was undertaken using a survey featuring a series of 25 lung ultrasound clips, sourced and labelled with agreement from three ultrasound fellowship trained physicians. The survey was distributed to 100 LUS-trained acute care physicians from across Canada. Respondents were asked to identify the findings in a series of LUS loops according to the presence B lines vs normal lung (A line pattern), the characteristics of the pleural line (smooth or irregular) as well as the cause of the lung ultrasound findings (hydrostatic pulmonary edema, non-COVID pneumonia or COVID pneumonia). Responses were compared to the true, expert-defined labels consistent with the data curation process described above. Since the data used for modelling did not include normal lungs, it was decided that four clips of normal lungs (A line pattern) were discarded from analysis of human performance.

In at least some embodiments, an explainability metric can be computed to visually explain model predictions. For example, in at least some embodiments, the Grad-CAM method may be used (see, e.g., Selvaraju R R, Cogswell M, Das A, Vedantam R, Parikh D, Batra D, Grad-CAM: Visual Explanations from Deep Networks via Gradient-based Localization [Internet], arXiv [cs,CV], 2016, Available from: http://arxiv, org/abs/1610,02391). Grad-CAM generally involves visualizing the gradients of the prediction of a particular image with respect to the activations of the final convolutional layer of the CNN. In particular, generating the visualization may involve taking the gradient of the predicted class with respect to the feature map outputted by the final convolutional layer, taking the mean of this gradient for each feature map channel, multiplying each channel in the feature map by this averaged gradient vector, then taking the channel-wise mean to get a 2D vector, which is then normalized and upsampled to the original image dimension. The resultant heatmap highlights the areas of the input image that were most contributory to the model's classification decision.

Experimental results for one example embodiment will now be described. In the example embodiment, the data extraction process resulted in 84 cases of COVID which, as part of an effort to balance the groups for unbiased training, led to extracting 78 cases of NCOVID and 81 cases of HPE. Further characteristics of the data are summarized in Table 2A.

TABLE 2A
Data profile for the 3 groups of lung ultrasound images used to train and test the
example neural network model (SS: Sonosite, MR: Mindray, Ph: Philips)

| | COVID | NCOVID | HPE |
|---|---|---|---|
| Number of cases | 84 | 78 | 81 |
| Number of loops | 185 | 224 | 191 |
| Number of still | 30419 | 44193 | 46769 |
| images | | | |
| Female Sex (%) | 50% | 40% | 55% |
| Age (yr) | 60.6 +/− 11.3 | 56.0 +/− 16.0 | 67.2 +/− 15.3 |
| Machines Models (%) | SS Edge 77.38 | SS X-Porte56.4 | SS Edge 76.9 |
| | SS X-porte 11.9 | SSEdge 41.0 | SS X-Porte 19.2 |
| | Ph Lumify 5.9 | MR M9 2.6 | MRM9 3.9 |
| | SS Edge-2 1.2 | | |
| | SS S-Cath 1.2 | | |
| Transducers (%) | Phased 95.3 | Phased array 98.7 | Phased array 92.3 |
| | Curvilinear 3.6 | Curvilinear 1.3 | Curvilinear 7.7 |
| | Linear 1.2 | | |
| Imaging Preset (%) | Abdominal 98.8 | Abdominal 97.4 | Abdominal 87.2 |
| | Venous 1.2 | Lung 2.6 | Cardiac 7.7 |
| | | | Lung 5.1 |
| Different | 12 | 43 | 45 |
| sonographers | | | |
| Date Range | March 2020-June 2020 | August 2017-March 2020 | October 2018-April 2020 |

A benchmarking survey was completed by 61 physicians with a median of 3-5 years of ultrasound experience the majority of whom had done at least a full, dedicated month of ultrasound training (80.3%) and who described their comfort with LUS use as “very comfortable” (72.1%). Table 2B sets out additional demographic information of the human benchmarking participants.

TABLE 2B
Demographic information of participants in human
benchmarking survey

| | Total number (%) |
|---|---|
| Total number of participants | 61 |
| [symbol] | |
| Attending Physician | 28 (45.9) |
| Sr. Resident | 33 (54.1) |
| [symbol] | |
| <1 | 3 (4.9) |
| 1-3 | 23 (37.7) |
| 3-5 | 22 (36.1) |
| 5-10 | 7 (11.5) |
| >10 | 6 (9.8) |
| [symbol] | |
| None | 0 |
| POCUS fellowship | 14 (23) |
| POCUS residency elective | 49 (80.3) |
| Live POCUS course | 43 (70.5) |
| Online POCUS course | 11 (18) |
| [symbol] | |
| Very comfortable, routine use | 44 (72.1) |
| Somewhat comfortable | 16 (26.2) |
| Limited comfort | 1 (1.6%) |
| Not comfortable with use | 0 |
| [symbol] | |
| Pneumothorax | 54 (88.5) |
| Pleural effusion | 61 (100) |
| Interstitial syndrome | 61 (100) |
| Consolidation (Pneumonia/atelectasis) | 52 (85.2) |
| [symbol] | |
| [symbol] | |
| [symbol] | |
| Yes | 45 (73.8) |
| No | 16 (26.2) |

The results of this survey highlighted that the physicians were adept at distinguishing the HPE class of B lines from COVID and NCOVID causes of B lines. For the COVID and NCOVID cases, however, significant variation and uncertainty was demonstrated. See Table 3 below.

TABLE 3
Confusion matrix for the survey responses from 61 physicians
classifying LUS images into their respective causes. Bracketed
numbers reflect classifications from the aggregated approach
used to calculate AUC

| | | Predicted | | |
|---|---|---|---|---|
| Physicians | | COVID | NCOVID | HPE |
| Actual | COVID | 173 (3) | 162 (3) | 34 (2) |
| | NCOVID | 177 (4) | 163 (1) | 30 (2) |
| | HPE | 138 (0) | 102 (0) | 302 (6) |

For at least one example embodiment, after the model was trained on the training set, its performance on the 10.1% of images that constituted the holdback data (test-2) was evaluated at both the image and encounter level. The model's prediction for an image is the probability vector p=[p_{COVID}, p_{NCOVID}, p_{SMOOTH}] obtained from the output of the softmax final layer, and the predicted class was taken to be argmax(p). Prediction for an encounter was considered to be p=[p_{COVID}, p_{NCOVID}, p_{SMOOTH}] where p_{c} is the average predicted probability for class c over the predictions for all images within that encounter. Encounter-level predictions were computed and presented to (1) replicate the method through which real time interpretation (by clinician or machine) occurs with ultrasound by aggregating images within one or more clips to form an interpretation and (2) closely simulate a physician's classification procedure, since the physicians who participated in the benchmarking survey were given entire clips to classify. In the example embodiments, the model demonstrated an ability to distinguish between the three classes in the dataset. Confusion matrices on the test-2 set at the frame and encounter level are depicted in Tables 4 and 5. Classification performance metrics are detailed in Table 6.

TABLE 4
Confusion matrix for model performance on the test-2
holdback set at the individual image (frame) level

| | | Predicted | | |
|---|---|---|---|---|
| CNN-Frames | | COVID | NCOVID | HPE |
| Actual | COVID | 3188 | 256 | 7 |
| | NCOVID | 1176 | 3741 | 3 |
| | HPE | 109 | 1119 | 2771 |

TABLE 5
Confusion matrix for model performance on the test-2
holdback set at the at the encounter level

| | | Predicted | | |
|---|---|---|---|---|
| CNN-Encounters | | COVID | NCOVID | HPE |
| Actual | COVID | 6 | 0 | 0 |
| | NCOVID | 1 | 6 | 0 |
| | HPE | 0 | 3 | 4 |

TABLE 6
Classification performance metrics calculated from the model's
predictions and ground truth from the test-2 set. Metrics are
reported at both the frame and encounter level. COVID: COVID-19
pneumonia, HPE: hydrostatic pulmonary edema, NCOVID: non-COVID
related acute respiratory distress syndrome.

| | | Sensi- | | | | |
|---|---|---|---|---|---|---|
| Prediction | | tivity/ | Speci- | Preci- | F1- | |
| Type | Class | Recall | ficity | sion | score | AUC |
| Frames | COVID | 0.924 | 0.883 | 0.713 | 0.805 | 0.965 |
| | NCOVID | 0.760 | 0.815 | 0.731 | 0.746 | 0.893 |
| | HPE | 0.693 | 0.999 | 0.996 | 0.817 | 0.991 |
| Encounters | COVID | 1.0 | 0.929 | 0.857 | 0.923 | 1.0 |
| | NCOVID | 0.857 | 0.769 | 0.667 | 0.75 | 0.934 |
| | HPE | 0.571 | 1.0 | 1.0 | 0.727 | 1.0 |

Although some example embodiments use a neural network trained to assess one image at a time (and may take an average of predictions over a single encounter), in some other embodiments, the neural network may be trained to ingest and process entire videos or clips of an encounter (or multiple encounters).

For at least one example embodiment, a comparison of the model to the physician performance relied on a threshold-independent metric, namely AUC. Since AUC measures a classifier's ability to rank observations, the raw survey data (in the form of classifications, not probabilities) was processed to permit an AUC computation by considering physician-predicted probability of a LUS belonging to a specific class as the proportion of physicians that assigned the LUS to that class.

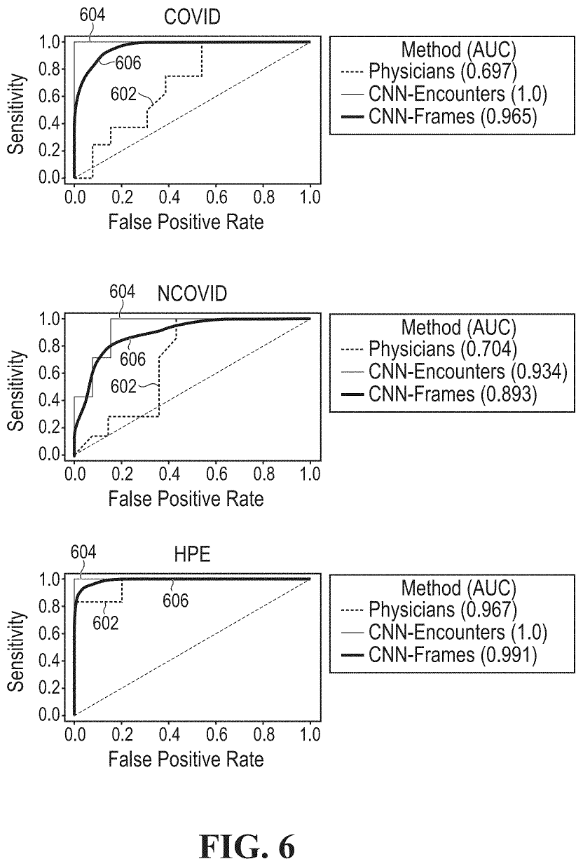

The physician AUCs were 0.697 (COVID), 0.704 (NCOVID), and 0.967 (HPE) for the results of the survey, leading to an overall AUC of 0.789. For the neural network (on the encounter level), the AUCs were 1.0 (COVID), 0.934 (NCOVID), and 1.0 (HPE), producing an overall AUC of 0.978. A comparison of the human and model AUCs is graphically illustrated in FIG. 6.

It is noted that the AUCs of approximately 0.7 for the physicians when the positive class is COVID or NCOVID is discordant with the raw data in the confusion matrix (Table 3), which suggests near random classification (which corresponds to an AUC of 0.5).

Although all three AUCs obtained by the physicians reached the 0.7 threshold associated with acceptable discrimination, the ROC curves and their associated scores suggest that physicians may have very limited—or no—ability to distinguish between COVID and NCOVID. All three AUCs appeared to be driven by the physicians' ability to separate HPE from the other two classes. When the positive class is either COVID or NCOVID, the top right part of the ROC curve exceeds random performance. The high level of performance associated with this section may have occurred because very few physicians diagnosed COVID/NCOVID ultrasounds as HPE. Consequently, even with a low threshold probability, HPE ultrasounds may have been identified as part of the negative class, reducing the false positive rate while the sensitivity remained high. This section of the curve therefore may have inflated the AUC above random performance. The physicians' ability to distinguish between COVID and NCOVID may be better represented by the bottom left part of the ROC curve. When either COVID or NCOVID is the positive class, the bottom part of the curve approximately follows along the 45° line associated with random performance.

In order to quantitatively validate the above qualitative observations, two different approaches may be used. Both are designed to remove HPE, converting the problem to a binary (COVID vs NCOVID) problem. The first approach is to remove physician diagnoses that are no longer relevant (in this case, HPE), and to replace these diagnoses with randomly generated diagnoses from the remaining candidates. The second approach is to condition the physicians' aggregate diagnosis on the knowledge that the ultrasound was not HPE. The AUCs obtained from these two procedures were 0.455 and 0.429 respectively, both below the 0.5 threshold associated with random performance.

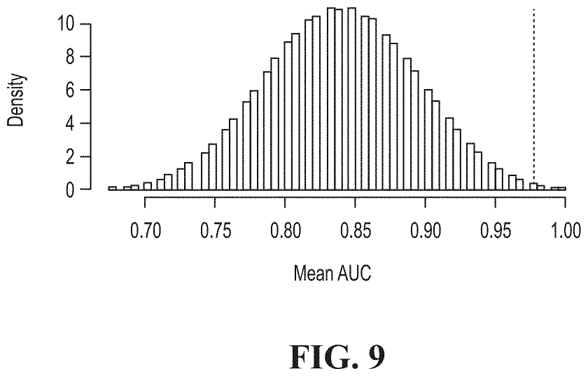

In order to perform a statistical test comparing the model's results to human performance, and in particular to test if the model was able to distinguish between COVID and NCOVID, a Monte Carlo Simulation (MCS) was performed (see, e.g., Andrieu C, de Freitas N, Doucet A, Jordan Mich., An Introduction to MCMC for Machine Learning, Mach Learn, 2003 Jan. 1; 50(1):5-43). For the MCS, a classifier was used that perfectly separated HPE from COVID and NCOVID (i.e., all HPE cases were predicted as HPE with a probability of one and all COVID/NCOVID cases were assigned zero probability of being HPE), but guessed when deciding between COVID and NCOVID. For the COVID and NCOVID cases, the predicted probability of the observation belonging to the COVID class was drawn from a Uniform(0, 1) distribution and the remaining probability was assigned to the NCOVID class. This classifier provided an upper bound on performance for a model unable to distinguish between COVID and NCOVID (i.e., an upper bound on human performance). Thus, if a model is shown to outperform this model, it may be assumed to be able to distinguish between COVID and NCOVID. The performance of this classifier was simulated on a testing dataset (e.g., test-2 dataset) one million times in order to obtain the distribution of its overall AUC. The results are shown in FIG. 9, as a histogram displaying the results of the MCS with one million runs, with the dashed vertical line representing the AUC of the neural network. After simulating this performance one million times, the MCS yielded an average AUC of 0.840 across all three classes, again suggesting that the example neural network model exceeds human performance, and in particular that the model can distinguish between COVID and NCOVID (p<0.01). The unusual distribution was caused by a correlation of 1.0 between the AUCs when COVID and NCOVID were the positive class.

Referring now to FIG. 6, there are illustrated receiver operating characteristic curves across the three classes of images that human benchmarking (physicians) 602 and an example neural network model (CNN) 604, 606 were tasked with interpreting. The model's performance on the test-2 (holdback) image set is plotted for both individual images 606 and across the entire image set from one encounter 604. In all image categories, it can be seen that the model interpretation accuracy exceeded that of the human interpretation and, in particular, exceeded human ability to distinguish between COVID and NCOVID ARDS.

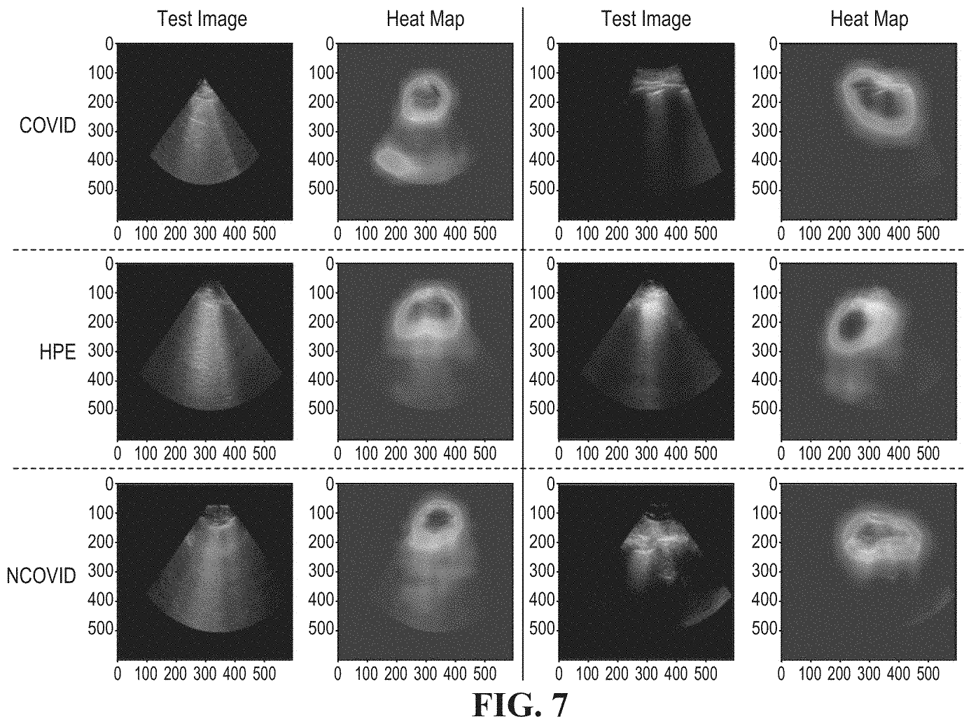

As described elsewhere herein, in at least some embodiments an explainability metric can be used. In the above-noted example neural network model, the Grad-CAM explainability approached was applied to the output from the model on the holdback data. The results are conveyed by shading on the heatmap, overlaid on test-2 input images. Referring now to FIG. 7, there are illustrated test images and the corresponding heatmaps for two example images in each of the three classes of image: HPE, COVID and NCOVID. Shading reflects the regions of the image with the highest contribution to the resulting class predicted by the model and, in particular, darker and lighter regions within the circle-shaped structures found within the wedge-shaped detection area correspond to highest and lowest prediction importance, respectively. As can be seen, the key activation areas for all classes were centered around the pleura and the pleural line. In all cases, the immediate area surrounding the pleura appears most activated.

At least some of the described embodiments appear to be capable of distinguishing the underlying pathology in similar point-of-care lung ultrasound images containing B lines. In particular, at least some of the described embodiments appear to be able to strongly distinguish COVID-19 from other causes of B lines and appear to outperform ultrasound trained clinician benchmarks across all categories. This suggests that subvisible, disease-specific, pixel-level, digital biomarker profiles exist within lung ultrasound images and can be detected using at least some of the described embodiments.

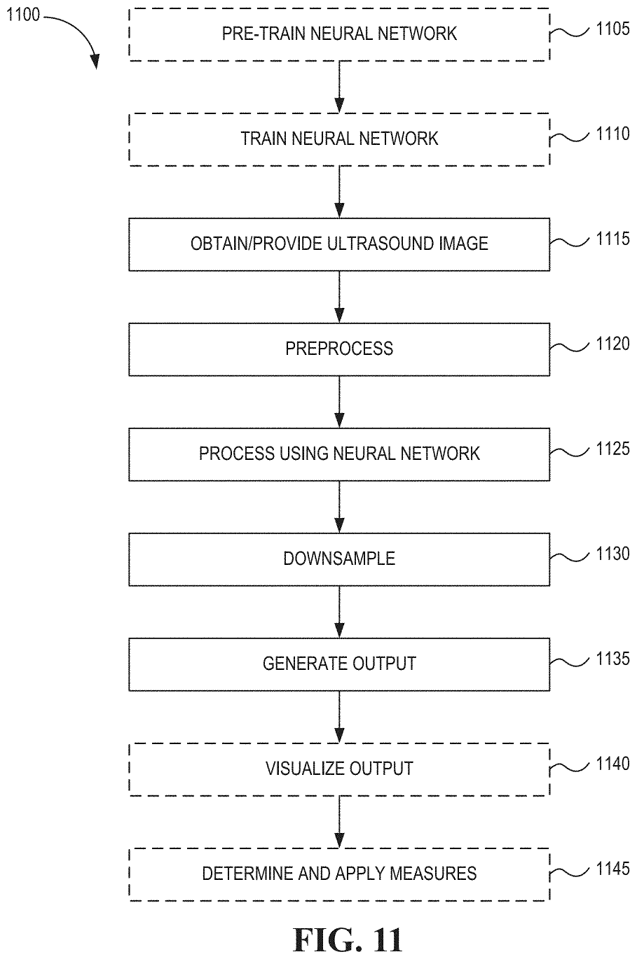

Referring now to FIG. 11, there is shown a process flow diagram for a method of processing data to distinguish between a plurality of conditions of a lung in accordance with at least some embodiments.

Method 1100 may be carried out, for example, using a computer system and ultrasound device such as those described elsewhere herein.

Method 1100 may optionally begin at 1105 with retrieving pre-trained weights, or by pre-training of the neural network to obtain pre-trained weights. For example, the neural network may be pre-trained by performing acts 1115 to 1135 as described further herein over a dataset of pre-training images from a database such as the ImageNet database. Following pre-training, or upon retrieving pre-trained weights, a “head” portion or first portion of the neural network may be discarded.

Next, at 1110, the neural network may be optionally trained using a training dataset of ultrasound images of lungs. Again, the neural network may be trained by performing acts 1115 to 1135 as described further herein over a validated dataset of ultrasound training images containing ultrasound images validated as belonging to a plurality of classes of interest. As a result of training, the neural network can be configured to activate in response to the presence of one or more subvisible artifact in the at least one ultrasound image and, in particular, to one or more subvisible artifact located in a pleural line region of the at least one ultrasound image. The ultrasound image may contain B lines, for example.

Training may be performed, for example, using an Adam optimizer with a learning rate of 1×10⁻⁶. Training may be further performed with early stopping with a patience of 3 epochs conditioned on validation loss. During training, the downsampling may further include applying dropout at a rate of between 0.1 to 0.9, preferably between 0.4 to 0.8, and still more preferably about 0.6. During training, the preprocessing may further include performing an augmentation transformation to the at least one ultrasound image, where the augmentation transformation can be any one or more of: random zooming by up to about 10%; horizontal flipping; horizontal stretching or contraction by up to about 20%; vertical stretching or contraction by up to about 5%; or rotation by up to about 10°.

Following training, at 1115 at least one ultrasound image of the lung may be provided for processing. In some cases, the at least one ultrasound image may be obtained from a subject, for example, via a point-of-care ultrasound device although other ultrasound devices may also be used. The at least one ultrasound image is preprocessed at 1120 to produce a tensor representation of the at least one ultrasound image. In some cases, the tensor may have dimensions of, e.g., 512×512×3, although other dimensions may be used.

In some embodiments, the at least one ultrasound image may be pre-processed by the acquisition device to produce a visual image suitable for review by a human practitioner. However, in some other embodiments, the at least one ultrasound image may be raw data without visual pre-processing. Use of such images may serve to enhance the accuracy of the neural network processing, since it may leave additional information in the image data.

Preprocessing may include converting the image to grayscale. In some cases, an RGB or other color image format may be retained even though the color information in the image may be converted to grayscale.

In some cases, the at least one ultrasound image may be a plurality of ultrasound images forming a video or clip of an encounter, in which case the preprocessing may include selecting one or more still images from the video. If individual images are processed separately, then at 1135, the aggregated outputs for the encounter may be averaged or otherwise combined to generate a combined output for the encounter. In some embodiments, however, the neural network may be trained using videos, in which case videos the at least one ultrasound image may be a video of an encounter.

At 1125, the tensor is processed using the neural network to produce an intermediate tensor representation. The neural network may be a depthwise separable convolutional neural network as described elsewhere herein, such as a modified Xception neural network.

At 1130, the intermediate tensor may be downsampled to produce an output tensor. Downsampling may involve performing two-dimensional global average pooling to produce a one-dimensional output tensor.

At 1135, an output tensor can be generated using an output neural network. In some cases, the output neural network is a 3-layer fully connected network with softmax activation.

The output tensor may represent a probability of the presence of a first condition of the plurality of conditions in the at least one ultrasound image. Further, generating the probability may further involve generating an output classification vector, where the output classification vector represents a plurality of probabilities of each of a plurality of conditions. For example, the first condition may be acute respiratory distress syndrome due to COVID-19, a second condition may be acute respiratory distress syndrome due to non-COVID-19 causes, and a third condition may be hydrostatic pulmonary edema.

Optionally, at 1140, a visualization, such as a gradient heatmap, may be generated that depicts activations of the final convolutional layer of the neural network to identify relative contributions of each portion of the at least one ultrasound to the probability, as described elsewhere herein. The gradient heatmap may be resampled to an original size of the at least one ultrasound image, and overlaid onto the at least one ultrasound image, to produce a validation image.

Optionally, at 1145, one or more additional acts may be taken if a probability of a condition (e.g., a first condition such as ARDS due to COVID-19) exceeds a predetermined threshold. For example, the predetermined threshold may be a 55-65% probability, or preferably a 65-75% probability, or still more preferably a 75-85% probability. In some cases, there may be a plurality of predetermined thresholds, which may represent graded classifications. For example, a 55-65% probability—or lower—may represent a low probability threshold, a 65-75% probability may represent a moderate probability threshold, a 75-85% probability may represent a high probability threshold, and a greater than 85% probability may represent a very high or extreme probability threshold.

When the predetermined threshold is met or exceeded, a subject may be isolated in accordance with a triage protocol. For example, in some cases, a greater than 85% probability of a first condition such as ARDS due to COVID-19 may be treated as equivalent to a positive polymerase chain reaction test for SARS-CoV-2. Alternatively, a low probability of less than 65% or 55% may be suggestive that SARS-CoV-2 infection is unlikely, leading to alternative diagnostic considerations (e.g., assessing for alternative causes of respiratory failure, etc.) and/or altering consumption of personal protective equipment.

Alternatively, or in addition, a first treatment may be selected and applied in response to the probability meeting or exceeding the predetermined threshold of a first condition, such as ARDS due to COVID-19. For example, the first treatment may include administration of one or more of: hemodynamic support (e.g., vasopressors such as norepinephrine), ventilatory support, extracorporeal membrane oxygenation, antiviral medication (e.g., remdesivir), immune-based therapy (e.g., COVID-19 convalescent plasma or SARS-CoV-2 immune globulins), or corticosteroids (e.g., dexamethasone).

Similarly, a second treatment may be selected and applied in response to the probability meeting or exceeding the predetermined threshold for a second condition, such as ARDS due to non-COVID causes. In this case, the second treatment may include performing additional diagnostics, such as respiratory cultures, to isolate the specific, non-COVID cause of ARDS. The second treatment may also include treatment with antibiotics.

Also similarly, a third treatment may be selected and applied in response to the probability meeting or exceeding the predetermined threshold for a third condition, such as HPE (e.g., caused by heart failure). In this case, the third treatment may include administration of diuretic therapy, blood pressure reduction therapies and diagnostic testing, including assessing for underlying cardiac and/or renal diseases.

In some cases, it can be presumed that received lung ultrasound clips exhibit B-line features, and accordingly, these can be analyzed to determine the cause (i.e., etiology) of the B-lines. In many cases, however, ultrasound clips may image normal lungs which do not exhibit B-line features. In these cases, there may be no necessity to further analyze these clips to determine underlying pathological causes. In view of the foregoing, prior to performing a diagnostic analysis of lung ultrasound clips using a B-line classifier, it may be beneficial to initially determine whether a lung ultrasound is imaging lungs in a pathological class (i.e., an image of abnormal lung parenchyma) or a normal class (i.e., an image of normal lung parenchyma). As such, only ultrasounds clips that image lungs in a pathological class may be subject to further analysis in order to classify the cause of the abnormality, i.e., in accordance with methods and processes described above.

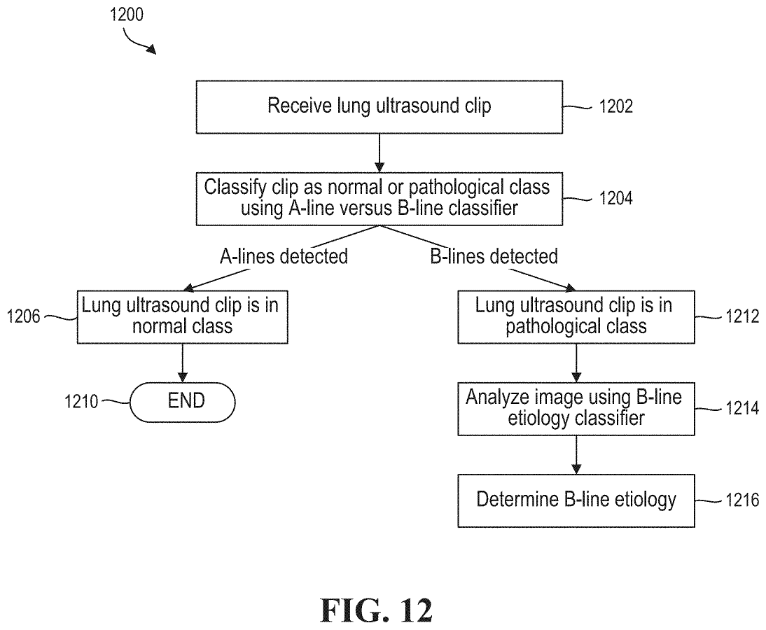

Reference is now made to FIG. 12, which shows an example embodiment of a process flow for a method 1200 for initially determining whether a lung ultrasound clip is part of a normal class or a pathological class. Method 1200 may be carried out, for example, using a computer system and ultrasound device such as those described elsewhere herein.

As shown, at 1202, a lung ultrasound clip (or image) is received for analysis. At 1204, the lung ultrasound clip is processed to initially classify the clip as being part of a normal class or a pathological class. In embodiments described herein, this classification is performed using an A-line versus B-line deep learning classifier. More particularly, while B-lines are vertical artifacts that are typically indicative of abnormal lungs, A-lines are horizontal artifacts that appear in ultrasound images and normally indicate a normal lung surface. Accordingly, in embodiments provided herein, an A-line versus B-line classifier may be used to determine whether imaged lungs, in an ultrasound clip, belong to a normal class (i.e., A-lines detected in the ultrasound clip) versus a pathological class (i.e., B-lines detected in the ultrasounds clip).

At 1206, if A-lines are detected by the classifier, then the ultrasound image is classified in a normal class, and at 1210, the method may end as no further analysis is required.

Alternatively, if B-lines are detected by the classifier, then at 1212, the ultrasound image is classified in a pathological class. Accordingly, at 1214, further diagnostic analysis is performed on the clip using a B-line etiology classifier in accordance with the methods and processes described elsewhere herein such as to allow the B-line etiology to be determined at 1216.

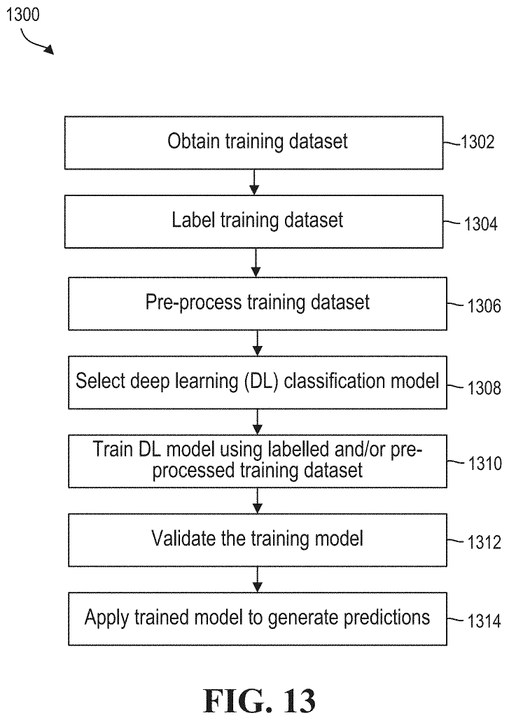

Reference is now made to FIG. 13, which shows an example embodiment of a process flow for a method 1300 for training and implementing an A-line versus B-line classifier. The classifier can be used at act 1204 in method 1200 to identify lung ultrasounds clips that include the presence of only A-lines, i.e., indicating normal lung parenchyma, versus the presence of B-lines, i.e., indicating abnormal parenchyma, so as to allow for rapidly constructing meaningful diagnosis of lung ultrasounds clips. Method 1300 may be carried out, for example, using a computer system and ultrasound device such as those described elsewhere herein.

At 1302, a training dataset is obtained to train the classifier model. The training dataset can include data obtained from an ultrasound database (i.e., an institutional point-of-care ultrasound database) and can include lung ultrasounds clips and/or images. In some cases, the lung ultrasounds clips are obtained in an MP4 format. In at least some cases, the lung ultrasounds clips and/or images are anonymized with masking of any information that is extraneous to the ultrasound beam (i.e. masking vendor name, depth markers, index markers, etc.). The masking may be performed using an application customized for deep learning and ultrasound (i.e., an application by WaveBase Inc.®). In some cases, the training clips and/or images are also pre-screened for appropriateness such that unsuitable clips and/or images are removed (i.e., images or clips with text over image, uninterpretable image compression or those of cardiac abdominal structures).

At 1304, the training dataset—comprising various lung ultrasound clips and/or images—are labeled for the presence of A-lines or B-lines. In some cases, the labelling may be performed manually, e.g., by a trained practitioner, using a labelling software or platform (i.e., Labelbox®). In some embodiments, clips and/or images labelled with B-lines can be labelled into three groups: (a) clips and/or images showing fewer than three B-lines in the field; (b) clips and/or images showing B-lines occupying less than 50% of the pleural lines and (c) clips and/or images showing B-lines occupying more than 50% of the pleural line surface. Clips and/or images can also be labeled for the presence of lung sliding, pleural thickness, subpleural consolidation, as well as labelling whether the clips are homogenous (e.g., B-lines present on screen throughout duration of clip) or heterogenous (e.g., B-lines emerging in and out of view with tidal respiration). In some cases, clips that include abdominal solid organs and/or diaphragm can be considered pleural views that are excluded from analysis while all other parenchymal views are included in the analysis.

At 1306, the labelled training dataset can be pre-processed. In some embodiments, at act 1306, training clips are deconstructed into their constituent frames. Following this, at least some of the frames may be subject to additional scrubbing of all on screen information that is extraneous to the ultrasound beam itself (e.g. vendor logos, battery indicators, index mark, depth markers). In some cases, this scrubbing is performed using a dedicated deep learning masking tool for ultrasound (AutoMask® of WaveBase Inc.®). Additionally, the masking tool may set all pixel values extra to the ultrasound beam to zero. In some cases, prior to being passed to the model, frames may also be resized to 128×128×3 tensors. The image frames may also be converted from red green blue (RGB) to BGR format, and the values of each channel may be further zero-centered to the means of ImageNet®.

In various cases, upon the assembly of a batch of images for training, transformations may be stochastically applied as a means of data augmentation. Examples of possible transformations to image frames can include rotation up to 45° clockwise or counterclockwise, vertical or horizontal width shifting by up to 10%, magnification by up to 10% inwards or outwards, shear by up to 10° counterclockwise, horizontal reflection, and brightness increase/decrease by up to 30%. In some cases, these methods may be applied to increase the heterogeneity of the training dataset as, despite the training dataset comprising a large quantity of frames, the number of distinct clips and patients may be comparatively less.

At 1308, a deep learning classification model is selected. Such a model can be trained using the assembled labelled and/or pre-processed training dataset. The determined classification model architecture may be, for example, anyone of a feedforward convulsion neural network (CNN), a residual CNN and a benchmark CNN.

In response to initial experimentation results on subsets of training datasets, it has been appreciated by the inventors that two effective models include (a) a residual CNN that is trainable from scratch, and (b) a network that includes a first few blocks of VGG 16 with weights pre trained on ImageNet®. Further, it has also been appreciated that among these two models, the VGG 16-based model is a frontrunner among possible candidate models. In at least some embodiments, at 1310, a VGG 16-based model having the following customized architecture is selected:

TABLE 7
Custom VGG 16 Architecture

| Layer (type) | Output Shape | Param # |
|---|---|---|
| input (InputLayer) | [(None, 128, 128, 3)] | 0 |
| block1_conv1 (Conv2D) | (None, 128, 128, 64) | 1792 |
| block1_conv2 (Conv2D) | (None, 128, 128, 64) | 36928 |
| block1_pool (MaxPooling2D) | (None, 64, 64, 64) | 0 |
| block2_conv1 (Conv2D) | (None, 64, 64, 128) | 73856 |
| block2_conv2 (Conv2D) | (None, 64, 64, 128) | 147584 |
| block2_pool (MaxPooling2D) | (None, 32, 32, 128) | 0 |
| block3_conv1 (Conv2D) | (None, 32, 32, 256) | 295168 |
| block3_conv2 (Conv2D) | (None, 32, 32, 256) | 590080 |
| block3_conv3 (Conv2D) | (None, 32, 32, 256) | 590080 |
| global_avgpool (GlobalAverag | (None, 256) | 0 |
| dropout (Dropout) | (None, 256) | 0 |
| output (Dense) | (None, 2) | 514 |

Total params: 1,736,002
Trainable params: 1,736,002
Non-trainable params: 0

At 1310, the labelled and/or pre-processed training dataset (i.e., generated at acts 1304 and 1306) may be fed into the model architecture selected at act 1308 in order to train the model. In some embodiments, using a VGG 16 model in accordance with the architecture exemplified in table 7, individual preprocessed frames may be fed into the network as a tensor with dimensions 128×128×3. The input tensors may be sequentially passed through the first 3 blocks (i.e. 10 layers) of the VGG 16. The blocks may each comprise a few convolutional layers followed by a max pooling layer. The 32×32×256 output of the final convolutional layer may be passed to a 2D global average pooling layer. From there, it may be subjected to dropout at a rate of 0.45, then finally to a 2-node fully connected layer with softmax activation.

In at least some embodiments, at act 1310, the VGG 16 model training may be split into two phases: (i) feature extraction, and (ii) fine-tuning. In feature extraction, all ten (10) VGG 16 layers are frozen. The final layer is trained for six (6) epochs using an Adam® optimizer with a learning rate of 0.0003. During the fine-tuning phase, the weights comprising the third VGG 16 block and output fully connected layer are allowed to train at a significantly lower learning rate. These layers may be trained for nine (9) epochs using the RMSProp® optimizer at a learning rate of 9.3×10-. The weights of the first two blocks of VGG 16 may be kept stagnant during this phase because as they are previously trained on ImageNet® to recognize low-level patterns in images, which also appear in lung ultrasound images. Once training is halted and the model weights yielding lowest loss on the validation set are restored, this may be considered the end of a single training experiment. The best performing set of model weights on the validation set may then be used to evaluate model performance on the test set.

In some embodiments, to maximize model performance, a hyperparameter search using Bayesian hyperparameter optimization may be conducted. The hyperparameters of interest may include the learning rates for both the feature extraction and fine-tuning phase of training, the dropout rate, and the layer at which to unfreeze all subsequent layers during fine-tuning.

At 1312, the trained model may be validated. In some cases, the validation may involve initially randomly splitting the labelled and/or pre-processed training into a training, validation and test set by patient ID. In other words, all clips obtained from each unique patient may be confined to a single—training, validation, or test—set without overlap.

In at least some embodiments, model performance of a dataset at act 1312 may be determined by conducting a 10-fold cross validation. The kth fold may constitute the test set for the kth training experiment, and the validation set may be randomly selected from remaining data (table 8, below, shows example splits). The averages of test set metrics across all folds and their standard deviations may be taken as indicative of the model's performance and consistency respectively.

TABLE 8
Example K-fold cross validation experiment data
distribution by patients, clips, and frames

| | | Train | | | Validation | | | Test | | |
|---|---|---|---|---|---|---|---|---|---|---|
| Fold | Class | Patients | Clips | Frames | Patients | Clips | Frames | Patients | Clips | Frames |
| 1 | A-Lines | 203 | 579 | 149882 | 28 | 89 | 23880 | 22 | 55 | 13010 |
| | B-Lines | 128 | 285 | 70581 | 9 | 25 | 5820 | 18 | 44 | 9718 |
| 2 | A-Lines | 207 | 579 | 149380 | 24 | 72 | 21900 | 22 | 72 | 15492 |
| | B-Lines | 120 | 270 | 65400 | 14 | 35 | 8938 | 21 | 49 | 11781 |
| 3 | A-Lines | 203 | 586 | 151562 | 22 | 62 | 17520 | 28 | 75 | 17690 |
| | B-Lines | 127 | 300 | 73159 | 14 | 20 | 4380 | 14 | 34 | 8580 |
| 4 | A-Lines | 198 | 572 | 146872 | 26 | 78 | 18600 | 29 | 73 | 21300 |
| | B-Lines | 132 | 305 | 74059 | 12 | 20 | 5580 | 11 | 29 | 6480 |
| 5 | A-Lines | 201 | 592 | 150052 | 28 | 74 | 19620 | 24 | 57 | 17100 |
| | B-Lines | 125 | 278 | 66499 | 13 | 24 | 5820 | 17 | 52 | 13800 |
| 6 | A-Lines | 200 | 557 | 143272 | 26 | 82 | 20340 | 27 | 84 | 23160 |
| | B-Lines | 131 | 305 | 74179 | 11 | 14 | 3420 | 13 | 35 | 8520 |
| 7 | A-Lines | 200 | 560 | 143752 | 24 | 75 | 19920 | 29 | 88 | 23100 |
| | B-Lines | 130 | 306 | 74959 | 13 | 23 | 4560 | 12 | 25 | 6600 |
| 8 | A-Lines | 200 | 581 | 148972 | 26 | 73 | 18840 | 27 | 69 | 18960 |
| | B-Lines | 127 | 296 | 72739 | 13 | 24 | 5700 | 15 | 34 | 7680 |
| 9 | A-Lines | 203 | 566 | 145792 | 28 | 87 | 24540 | 22 | 70 | 16440 |
| | B-Lines | 127 | 301 | 72319 | 11 | 29 | 7380 | 17 | 24 | 6420 |
| 10 | A-Lines | 206 | 582 | 149272 | 24 | 61 | 16980 | 23 | 80 | 20520 |
| | B-Lines | 125 | 298 | 72859 | 13 | 28 | 6720 | 17 | 28 | 6540 |

In respect of validating frame level prediction—the model's prediction is a probability distribution indicating its confidence that an input lung ultrasound frame exhibits A-lines or B-lines. Predictions may be made for individual frames as opposed to whole clips as, despite being dynamic artifacts, single LUS frames are able to convey A vs B line patterns and may represent the building block unit of clips. Therefore, a classifier at the frame level may have the greatest agility to be applied to clips of varying compositions as is typical of point-of-care imaging.

In respect of clip level prediction—though the classifier may be trained to predict A vs B line at the frame level, the clinical application of LUS is typically dynamic—i.e., evaluated in real time or through recorded loops, as a series of sequential frames.

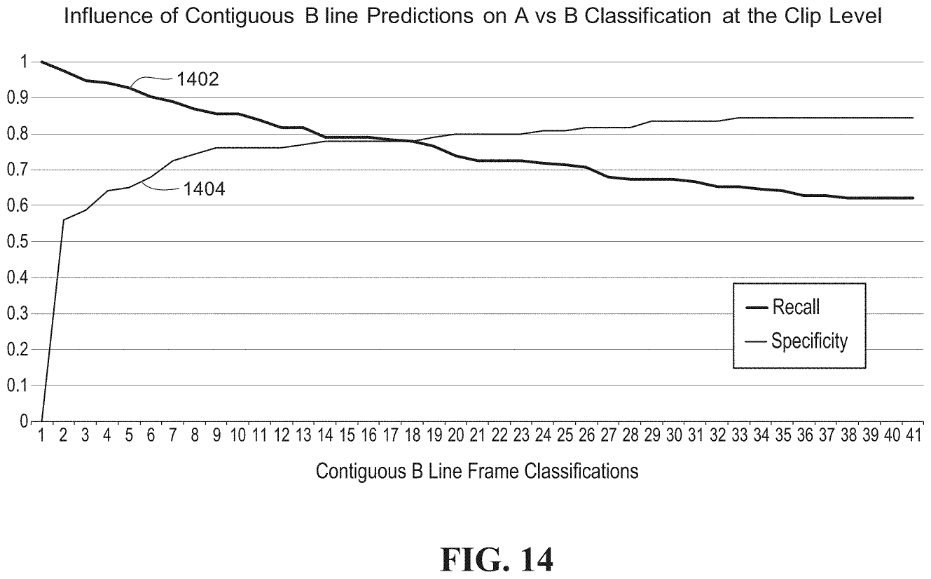

In considering clip level labels, a clinician performing LUS may regard most pathological feature(s) displayed within a clip, even if for a minority of the frames within the clip, to be the ground truth of said clip. A common example would be an A line pattern interrupted by a series of pathological B lines moving through the acoustic window due to tidal breathing. Accordingly, in embodiments provided herein, a clip level prediction is devised to capture this reality but also protect against volatility or weak predictions within the frame based model. That is, guarding against falsely positive B line labels at the clip level in the event of a small number of false positive B line frames within a set of several hundred A line frames. Thus a clip level prediction is provided for B lines that requires a minimum number of consecutively identified B lines at the frame level. The performance of the classifier may be modified to suit the desired sensitivity and specificity according to the clinical environment by applying and/more modifying the contiguity threshold. For scenarios where minimizing false negatives (higher recall for B lines) is desirable, a low contiguity threshold would be indicated (e.g. a threshold of 1) whereas the assurance of true positives is desirable (higher specificity for B lines, e.g. a contiguity threshold of 10-15). Referring briefly to FIG. 14, which shows a plot of contiguous B line predictions on A v B classification at a clip level. Plot line 1402 shows recall performance, while plot line 1404 shows specificity performance. As shown, the optimal threshold yielding the most stable performance was approximately between 13 and 15 B-line frames in the exemplified case. Values outside of this range exhibit a tradeoff between positive predictive value (i.e. precision) and sensitivity (i.e. recall).

In some embodiments, a Grad-CAM® method may be used to visualize which components of an input image are most contributory to the model's predictions. The product of Grad-CAM® is a heatmap overlaid onto the original image that highlights the regions of image that resulted in the highest activations of the model when predicting a certain class. In various cases, activated regions that coincide with the image characteristics that drive human classification may build clinician and investigator confidence in the fashion in which the neural network is learning.

Tables 9 to 12, below, demonstrate various validation results of an example trained VGG 16 based model.

Table 9 shows a k-fold cross validation of the trained model based on a first dataset. In this example case, the mean AUC across all folds was 0.9705 with a standard deviation of 0.0210. Individual AUC values and other metrics (accuracy, precision, recall, and F1 score) are broken down in table 9 by fold and class.

TABLE 9
Metrics for a 10-fold cross validation experiment on local data

| | | | F1 Score | F1 Score | Precision |
|---|---|---|---|---|---|
| Fold | Accuracy | AUC | (A lines) | (B lines) | (A lines) |
| 1 | 0.874076008796692 | 0.939964294433594 | 0.89068824 | 0.85150975 | 0.885211050510407 |
| 2 | 0.856048107147217 | 0.931204080581665 | 0.87296146 | 0.8339396 | 0.875227093696594 |
| 3 | 0.917776942253113 | 0.962266266345978 | 0.9387998 | 0.8747536 | 0.941092908382416 |
| 4 | 0.954319655895233 | 0.989018023014069 | 0.9703151 | 0.9009445 | 0.966944873332977 |
| 5 | 0.926828503608704 | 0.974798262119293 | 0.93556386 | 0.91535324 | 0.91244649887085 |
| 6 | 0.962847232818604 | 0.993862926959991 | 0.97487247 | 0.9287487 | 0.964148461818695 |
| 7 | 0.954377114772797 | 0.983974516391754 | 0.970678 | 0.8972629 | 0.970446944236755 |
| 8 | 0.921846866607666 | 0.978502810001373 | 0.9450544 | 0.8646998 | 0.945753216743469 |
| 9 | 0.94702535867691 | 0.986402750015259 | 0.96386695 | 0.90077835 | 0.945944368839264 |
| 10 | 0.89593493938446 | 0.964933574199676 | 0.93274426 | 0.77012247 | 0.914613604545593 |
| Mean | 0.92110807299614 | 0.970492750406265 | 0.9395545 | 0.87381124 | 0.932182902097702 |
| Standard | 0.036127195032921 | 0.021031989248052 | 0.0344303 | 0.04676139 | 0.033822696148339 |
| Deviation | | | | | |

| | Precision | Recall | Recall |
|---|---|---|---|
| Fold | (B lines) | (A lines) | (B lines) |
| 1 | 0.858727514743805 | 0.896233677864075 | 0.844412446022034 |
| 2 | 0.831127226352692 | 0.870707452297211 | 0.836771070957184 |
| 3 | 0.87041312456131 | 0.936517834663391 | 0.879137516021729 |
| 4 | 0.911546349525452 | 0.973708927631378 | 0.89058643579483 |
| 5 | 0.946866989135742 | 0.959883034229279 | 0.885869562625885 |
| 6 | 0.95899486541748 | 0.985837638378143 | 0.900352120399475 |
| 7 | 0.898011863231659 | 0.970909118652344 | 0.896515130996704 |
| 8 | 0.863129198551178 | 0.944356560707092 | 0.866276025772095 |
| 9 | 0.950216054916382 | 0.982481777667999 | 0.856230556964874 |
| 10 | 0.826094567775726 | 0.951608180999756 | 0.72125381231308 |
| Mean | 0.891512775421143 | 0.947224420309067 | 0.857740467786789 |
| Standard | 0.049170035083882 | 0.037644051602081 | 0.052671159281185 |
| Deviation | | | |

Table 10 shows frame based performance validation of the trained model based on a second dataset. The AUC obtained from the data at the frame level was 0.927. Table 10 provides a summary of metrics (precision, recall, accuracy, AUC) obtained on the data. The confusion matrix (table 11) of frame-wise predictions exhibit a strong diagonal pattern, supporting the results of the individual class performance.

TABLE 10
External Data Frame-Based Metrics

| | A_lines | B_lines |
|---|---|---|
| Precision | 0.8404255319148937 | 0.8773584905660378 |
| Recall | 0.8586956521739131 | 0.8611111111111112 |
| F1 Score | 0.849462365591398 | 0.869158878504673 |
| Accuracy | 0.86 | |
| AUC | 0.927133655394525 | 0.927133655394525 |

| | |
|---|---|
| Macro_Mean AUC | 0.927133655394525 |
| Weighted_Mean AUC | 0.927133655394525 |

TABLE 11
Confusion Matrix for external data frame-based performance

| | | Predicted | | |
|---|---|---|---|---|
| CNN-Frames | | A_lines | B_lines | Total |
| Actual | A_lines | 9439 | 1313 | 10752 |
| | B_lines | 2367 | 10220 | 12587 |
| | Total | 11806 | 11533 | |

Returning back to FIG. 13, after generating the trained model, at 1314, the trained model is used to generate predictions in respect of new ultrasounds lung clips and/or images to determine whether the clips and/or images correspond to a normal class (i.e., clips and/or images exhibiting A-lines) or a pathological class (i.e., clips and/or images exhibiting B-lines).

The present invention has been described here by way of example only, while numerous specific details are set forth herein in order to provide a thorough understanding of the exemplary embodiments described herein. However, it will be understood by those of ordinary skill in the art that these embodiments may, in some cases, be practiced without these specific details. In other instances, well-known methods, procedures and components have not been described in detail so as not to obscure the description of the embodiments. Various modification and variations may be made to these exemplary embodiments without departing from the spirit and scope of the invention, which is limited only by the appended claims.

1. A method of processing data to distinguish between a plurality of conditions based on at least one ultrasound image of a lung, the method comprising:
providing the at least one ultrasound image of the lung;
preprocessing the at least one ultrasound image to produce a tensor;
processing the tensor using a neural network to produce an intermediate tensor;
downsampling the intermediate tensor to produce an output tensor; and
processing the output tensor using an output neural network to generate a probability of presence of a first condition of the plurality of conditions in the at least one ultrasound image.

2. (canceled)

3. The method of claim 1, wherein the at least one ultrasound image is a plurality of ultrasound images forming a video, and wherein the preprocessing further comprises selecting a still image from the video for use in the tensor.

4. The method of claim 1, wherein the neural network is a depthwise separable convolutional neural network.

5. (canceled)

6. (canceled)

7. The method of claim 1, wherein the downsampling comprises performing two-dimensional global average pooling, and wherein the output tensor is one-dimensional.

8. (canceled)

9. The method of claim 1, wherein generating the probability further comprises generating an output classification vector, wherein the output classification vector represents a plurality of probabilities of each of the plurality of conditions.

10. The method of claim 1, wherein the first condition is acute respiratory distress syndrome due to COVID-19 and
wherein a second condition of the plurality of conditions is acute respiratory distress syndrome due to non-COVID-19 causes.

11. (canceled)

12. The method of claim 6, wherein a third condition of the plurality of conditions is hydrostatic pulmonary edema.

13. (canceled)

14. (canceled)

15. The method of claim 1, wherein the at least one ultrasound image contains B-lines.

16. The method any claim 1, further comprising determining that the probability of the first condition exceeds a predetermined threshold.

17. The method of claim 9, further comprising isolating a subject in accordance with a triage protocol in response to determining that the probability of the first condition exceeds the predetermined threshold.

18. The method of claim 10, further comprising applying a first treatment to a subject in response to determining that the probability of the first condition exceeds the predetermined threshold.

19. The method of claim 1, wherein the at least one ultrasound image is obtained via a point-of-care ultrasound device.

20. The method of claim 1, further comprising, prior to processing the tensor, pre-training the neural network to obtain pre-trained weights by performing the obtaining the at least one ultrasound image, the preprocessing, the processing the tensor, the downsampling and the processing the output tensor, wherein, during pre-training, the at least one ultrasound image is obtained from an image database.

21. (canceled)

22. (canceled)

23. The method of claim 13, further comprising training the neural network to obtain trained weights by performing the obtaining the at least one ultrasound image, the preprocessing, the processing the tensor, the downsampling and the processing the output tensor, wherein, during training, the at least one ultrasound image is obtained from a validated ultrasound image dataset.

24. (canceled)

25. (canceled)

26. The method of claim 14, wherein, during training, the downsampling further comprises applying dropout at a rate of about 0.6.

27. The method of claim 14, wherein, during training, the preprocessing further comprises performing an augmentation transformation to the at least one ultrasound image.

28. The method of claim 17, wherein the augmentation transformation is selected from the group consisting of: random zooming by up to about 10%; horizontal flipping; horizontal stretching or contraction by up to about 20%; vertical stretching or contraction by up to about 5%; or rotation by up to about 10°.

29. (canceled)

30. (canceled)

31. (canceled)

32. The method of claim 1, further comprising obtaining the at least one ultrasound image of the lung from a subject and initially analyzing the at least one ultrasound image using an A-line versus B-line deep learning classifier to determine whether the at least one ultrasound image corresponds to a pathological class.

33-45. (canceled)

46. A non-transitory computer readable medium storing computer program instructions which, when executed by at least one processor, cause the at least one processor to carry out a method of processing data to distinguish between a plurality of conditions based on at least one ultrasound image of a lung, the method comprising:
providing the at least one ultrasound image of the lung;
preprocessing the at least one ultrasound image to produce a tensor;
processing the tensor using a neural network to produce an intermediate tensor;
downsampling the intermediate tensor to produce an output tensor; and
processing the output tensor using an output neural network to generate a probability of presence of a first condition of the plurality of conditions in the at least one ultrasound image.

47. A system for processing data to distinguish between a plurality of conditions based on at least one ultrasound image of a lung, the system comprising:
a memory; and
at least one processor configured to:
obtain the at least one ultrasound image of the lung;
preprocess the at least one ultrasound image to produce a tensor;
process the tensor using a neural network to produce an intermediate tensor;
downsample the intermediate tensor to produce an output tensor; and
process the output tensor using an output neural network to generate a probability of presence of a first condition of the plurality of conditions in the at least one ultrasound image.

48. (canceled)
